SEQUENCE LISTING (4 sequences)

SEQ ID NO: 1 - protein, 411 aa, synthetic construct
TPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQLWAGLRATLPPTQEALPSSHSSPQQQGGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 106 aa, synthetic construct
MHHHHHHGMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGG

SEQ ID NO: 3 - DNA, 1236 bp, synthetic construct
  source 1..1236: mol_type = other DNA
acacctgtctcgcagaccaccacagctgccactgcctcagttagaagcacaaaggacccctgcccctcccagcccccagtgttcccagcagctaagcagtgtccagcattggaagtgacctggccagaggtggaagtgccactgaatggaacgctgagcttatcctgtgtggcctgcagccgcttccccaacttcagcatcctctactggctgggcaatggttccttcattgagcacctcccaggccgactgtgggaggggagcaccagccgggaacgtgggagcacaggtacgcagctgtgcaaggccttggtgctggagcagctgacccctgccctgcacagcaccaacttctcctgtgtgctcgtggaccctgaacaggttgtccagcgtcacgtcgtcctggcccagctctgggctgggctgagggcaaccttgccccccacccaagaagccctgccctccagccacagcagtccacagcagcagggtggtggtggtggttctggtggtggtggatctggtggtggaggttctgaaccaaagtcttgtgataagactcacacttgtccaccatgtccagctcctgaacttctgggtggaccatctgtctttcttttcccaccaaaacctaaggacactcttatgatttcccgtactcctgaagtcacttgtgttgttgtggacgtgagtcacgaagaccctgaggtcaagttcaactggtacgttgacggtgttgaagttcataatgccaagactaagcctcgtgaagagcaatacaacagtacttaccgtgttgtcagtgtccttaccgtcctgcaccaggactggctgaatggtaaggagtacaagtgtaaggtctccaacaaggcccttccagccccaatcgagaagaccatctccaaagccaagggtcaaccacgtgaaccacaagtttacaccctgcctccatcccgtgaggagatgaccaagaaccaggtcagtctgacttgtctggtcaagggtttctatccttccgacatcgctgttgagtgggagtccaacggtcaaccagaaaacaactacaagaccacccctccagttcttgactccgacggttccttcttcctttactccaagcttaccgttgacaagtccagatggcaacaaggtaacgttttctcatgttccgttatgcacgaagctctgcacaaccactacactcaaaagagcctttccctgtccccaggtaagtaa

SEQ ID NO: 4 - DNA, 318 bp, synthetic construct
  source 1..318: mol_type = other DNA
atgcatcatcatcatcatcacggcatgtcggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaattcaagctgatcagacccctgaagatttggacatggaggataacgatatcattgaggctcacagagaacagattggtggt

(19) United States
(12) Patent Application Publication
(10) Pub. No.: US 2023/0407364 A1
(43) Pub. Date: Dec. 21, 2023

(54) LONG-ACTING RECOMBINANT INTERLEUKIN-18 BINDING PROTEIN AND PRODUCTION METHOD AND APPLICATION THEREOF

(71) Applicant: Guangdong Medical University, Dongguan (CN)

(72) Inventors: Yechen Xiao, Dongguan (CN); Lei Guo, Dongguan (CN); Xiuze Chen, Dongguan (CN)

(21) Appl. No.: 18/332,489

(22) Filed: Jun. 9, 2023

(30) Foreign Application Priority Data
Jun. 16, 2022 (CN) 202210685070X

(51) Int. Cl. C12P 21/02; C07K 16/24; C12N 15/70

(52) U.S. Cl. CPC C12P 21/02; C07K 16/244; C12N 15/70; C12N 2800/101

(57) ABSTRACT

A long-acting recombinant interleukin-18 binding protein (IL-18BP) and its production method and application are provided, belonging to the field of biotechnology. The recombinant IL-18BP includes a sequence of human IL-18BP isoform a and a sequence of human IgG-Fc, and an amino acid sequence of the recombinant IL-18BP is shown in SEQ ID NO: 1. In the preparation method of the recombinant IL-18BP, an encoding gene expressing a fusion protein is inserted into a cell expression vector and the vector is introduced into prokaryotic cells to express the recombinant IL-18BP. A prokaryotic expression system is used to express the recombinant IL-18BP, promoting correct protein folding, enhancing protein stability, reducing in vivo degradation rate, and enhancing its biological activity.

### TECHNICAL FIELD

The disclosure relates to the field of biotechnologies, and more particularly to a long-acting recombinant interleukin-18 binding protein (IL-18BP) and a production method and an application thereof.

### STATEMENT REGARDING SEQUENCE LISTING

The sequence listing associated with this application is provided in text format in lieu of a paper copy and is hereby incorporated by reference into the specification. The name of the XML file containing the sequence listing is 23003TLAN-USP1-SL.xml. The XML file is 6,477 bytes; is created on Jun. 6, 2023; and is being submitted electronically via EFS-Web.

### BACKGROUND

Interleukin-18 (IL-18) is a cytokine produced by activated monocytes and dendritic cells, and has a variety of biological functions. IL-18 and IL-12 can synergistically induce natural killer (NK) cells and T helper 1 (Th1) cells to produce interferon-gamma (IFN-γ). IL-18 can enhance the cytotoxic effects of the Th1 and NK cells mediated by Fas, thereby exerting anti-infective and anti-tumor activities. In addition to mediating Th1 response, IL-18 is also a macrophage activator that can trigger the production of various proinflammatory cytokines, including tumor necrosis factor-alpha (TNF-α), IL-1, chemokine IL-8, and macrophage inflammatory protein-1 (MIP-1). Many studies have reported that the expression of IL-18 is significantly increased in some inflammatory diseases and autoimmune diseases, and it is a potential diagnostic marker of the diseases.

Interleukin-18 binding protein (IL-18BP) is a constitutively secreted protein and is a natural antagonist of IL-18, which can bind to IL-18 and neutralize its activity, thereby downregulating Th1 cytokine response, reducing the production of IFN-γ, and blocking the biological activity of IL-18. However, IL-18BP has no complete transmembrane structure and only has an immunoglobulin (Ig) domain. Among four isoforms of human IL-18BP (also referred to as hIL-18BPa, b, c and d), hIL-18BPa and hIL-18BPc have complete Ig domains and are therefore capable of binding to IL-18. In this situation, the hIL-18BPa is the isoform with the strongest binding capacity.

At present, the recombinant IL-18BP or IL-18BP fusion proteins are prepared by using eukaryotic cells, such as monkey COS-7 cells (also referred to as African green monkey kidney fibroblast cells), Chinese hamster ovary (CHO) cells (Raffaella Faggioni, 2001), and SF9 insect cells, but rarely expressed in prokaryotic cells. As a eukaryotic secretory protein, IL-18BP is easy to form an inactive inclusion body structure when expressed in a prokaryotic system. The inclusion bodies need to be denatured and renatured in the later stage, which will damage the activity of the recombinant protein to a certain extent. Due to the problem of renaturation efficiency of the inclusion bodies, it will also affect the purity of the protein in the later stage, thereby affecting the safety of the medication. Therefore, it is necessary to ensure the biological activity of the recombinant protein while also meeting the cost considerations for future large-scale production.

### SUMMARY

A purpose of the disclosure is to provide a long-acting recombinant interleukin-18 binding protein (IL-18BP) and its production method and application to solve the problems existing in the related art. By using a prokaryotic expression system to express the recombinant IL-18BP, the production of the recombinant protein is increased, and the production cost is greatly reduced.

In order to achieve the above purpose, the disclosure provides the following technical solutions.

The disclosure provides a recombinant IL-18BP, which includes a sequence encoding human IL-18BP isoform a and a sequence encoding human immunoglobulin class G-crystallizable fragment (IgG-Fc). The amino acid sequence of the recombinant IL-18BP is shown in SEQ ID NO: 1 as follows:

TPVSQTTTAATASVRSTKDPCPSQPPVFPAAKQCPALEVTWPEVEVPLNG
TLSLSCVACSRFPNFSILYWLGNGSFIEHLPGRLWEGSTSRERGSTGTQL
CKALVLEQLTPALHSTNFSCVLVDPEQVVQRHVVLAQLWAGLRATLPPTQ
EALPSSHSSPQQQGGGGGSGGGGSGGGGSEPKSCDKTHTCPPCPAPELLG
GPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHY
TQKSLSLSPGK.

In an embodiment, the recombinant IL-18BP includes a modified protein Sumo, and the amino acid sequence of the modified protein Sumo is shown in SEQ ID NO: 2 as follows:

MHHHHHHGMSDSEVNQEAKPEVKPEVKPETHINLKVSDGSSEIFFKIKKT
TPLRRLMEAFAKRQGKEM DSLRFLYDGIRIQADQTPEDLDMEDNDIIEA
HREQIGG.

In an embodiment, a coding gene for the modified protein Sumo is shown in SEQ ID NO:

4 as follows:

ATGCATCATCATCATCATCACGGCATGTCGGACTCAGAAGTCAATCAAGA
AGCTAAGCCAGAGGTCAAGCCAGAAGTCAAGCCTGAGACTCACATCAATT
TAAAGGTGTCCGATGGATCTTCAGAGATCTTCTTCAAGATCAAAAAGACC
ACTCCTTTAAGAAGGCTGATGGAAGCGTTCGCTAAAAGACAGGGTAAGGA
AATGGACTCCTTAAGATTCTTGTACGACGGTATTAGAATTCAAGCTGATC
AGACCCCTGAAGATTTGGACATGGAGGATAACGATATCATTGAGGC TCA
CAGAGAACAGATTGGTGGT.

The disclosure also includes a recombinant plasmid, including a coding gene expressing the recombinant IL-18BP, and the nucleotide sequence of the coding gene is shown in SEQ ID NO: 3 as follows:

ACACCTGTCTCGCAGACCACCACAGCTGCCACTGCCTCAGTTAGAAGCAC
AAAGGACCCCTGCCCCTCCCAGCCCCCAGTGTTCCCAGCAGCTAAGCAGT
GTCCAGCATTGGAAGTGACCTGGCCAGAGGTGGAAGTGCCACTGAATGGA
ACGCTGAGCTTATCCTGTGTGGCCTGCAGCCGCTTCCCCAACTTCAGCAT
CCTCTACTGGCTGGGCAATGGTTCCTTCATTGAGCACCTCCCAGGCCGAC
TGTGGGAGGGGAGCACCAGCCGGGAACGTGGGAGCACAGGTACGCAGCTG
TGCAAGGCCTTGGTGCTGGAGCAGCTGACCCCTGCCCTGCACAGCACCAA
CTTCTCCTGTGTGCTCGTGGACCCTGAACAGGTTGTCCAGCGTCACGTCG
TCCTGGCCCAGCTCTGGGCTGGGCTGAGGGCAACCTTGCCCCCCACCCAA
GAAGCCCTGCCCTCCAGCCACAGCAGTCCACAGCAGCAGGGTGGTGGTGG
TGGTTCTGGTGGTGGTGGATCTGGTGGTGGAGGTTCTGAACCAAAGTCTT
GTGATAAGACTCACACTTGTCCACCATGTCCAGCTCCTGAACTTCTGGGT
GGACCATCTGTCTTTCTTTTCCCACCAAAACCTAAGGACACTCTTATGAT
TTCCCGTACTCCTGAAGTCACTTGTGTTGTTGTGGACGTGAGTCACGAAG
ACCCTGAGGTCAAGTTCAACTGGTACGTTGACGGTGTTGAAGTTCATAAT
GCCAAGACTAAGCCTCGTGAAGAGCAATACAACAGTACTTACCGTGTTGT
CAGTGTCCTTACCGTCCTGCACCAGGACTGGCTGAATGGTAAGGAGTACA
AGTGTAAGGTCTCCAACAAGGCCCTTCCAGCCCCAATCGAGAAGACCATC
TCCAAAGCCAAGGGTCAACCACGTGAACCACAAGTTTACACCCTGCCTCC
ATCCCGTGAGGAGATGACCAAGAACCAGGTCAGTCTGACTTGTCTGGTCA
AGGGTTTCTATCCTTCCGACATCGCTGTTGAGTGGGAGTCCAACGGTCAA
CCAGAAAACAACTACAAGACCACCCCTCCAGTTCTTGACTCCGACGGTTC
CTTCTTCCTTTACTCCAAGCTTACCGTTGACAAGTCCAGATGGCAACAAG
GTAACGTTTTCTCATGTTCCGTTATGCACGAAGCTCTGCACAACCACTAC
ACTCAAAA GAGCCTTTCCCTGTCCCCAGGTAAGTAA.

The disclosure also provides a host bacterium, including the recombinant plasmid.

The disclosure also provides a method for preparing the recombinant IL-18BP, inserting the coding gene expressing the recombinant IL-18BP into a cell expression vector, and introducing the cell expression vector into prokaryotic cells to express the recombinant IL-18BP.

In an embodiment, the expression vector includes pET-20b(+).

The disclosure also provides a method for producing the recombinant IL-18BP by fermentation, including the step of obtaining the recombinant IL-18BP by inducing fermentation to culture the host bacterium.

In an embodiment, the expression conditions for isopropyl-β-D-thiogalactoside (IPTG) induction are as follows: induction at 0.5 millimoles per liter (mmol/L) IPTG at 20° C. for 4-31 hours.

The disclosure also provides a pharmaceutical composition, including the recombinant IL-18BP.

The disclosure also provides an application of the recombinant IL-18BP in preparation of drugs for treating inflammatory bowel disease.

The disclosure discloses the following technical effects.

In the disclosure, by using the folding promoting effect of molecular chaperone Sumo and the stability promoting effect of crystallizable fragment (Fc) tag, the method for inducing, expressing and purifying a soluble long-acting recombinant protein in the prokaryotic system is established, and a fermentation process system that can be used for large-scale production is optimized. The optimization experiments show that in a shaking flask, the expression level of the target protein Sumo-IL-18BP-Fc is the highest after induction at 0.5 mmol/L IPTG and 30° C. for 5 hours. In a fermentation tank, the expression level of the target protein is the highest after induction at 0.5 mmol/L IPTG and 20° C. for 26 hours, and the soluble expression level of the target protein after fermentation accounted for more than 85% of the total protein. The results of animal experiments show that the purified recombinant protein has high biological activity in both in vivo and in vitro. Therefore, through the prokaryotic expression system, the fusion protein disclosed by the disclosure can promote correct folding of the protein, enhance the stability of the protein, reduce the degradation rate in vivo, and enhance its biological activity. In addition, the yield of the recombinant protein can be increased through fermentation, the production cost is much lower than that of a eukaryotic cell expression system, which is conducive to industrial production.

### BRIEF DESCRIPTION OF DRAWINGS

In order to describe embodiments of the disclosure or technical solutions in the related art clearer, a brief introduction will be given to the accompanying drawings required in the embodiments. Apparently, the accompanying drawings in the following description are only some of the embodiments of the disclosure. For those skilled in the art, other accompanying drawings can also be obtained based on these drawings without any creative effort.

FIGS. 1A-1B illustrate that Sumo promotes soluble expression of interleukin-18 binding protein-crystallizable fragment (IL-18BP-Fc) protein. Specifically, in FIG. 1A, lane M represents a protein marker, lane 1 represents an empty vector pET-20b(+), lane 2 represent a recombinant Sumo-IL-18BP-Fc whole cell after induction at 37° C., lane 3 represents a protein supernatant of the recombinant Sumo-IL-18BP-Fc after fragmentation, lane 4 represents a protein precipitation of the recombinant Sumo-IL-18BP-Fc after fragmentation. In FIG. 1B, lane M represents a protein marker, lane 1 represents an empty vector pET-20b(+), lane 2 represent a recombinant IL-18BP-Fc whole cell after induction at 37° C., lane 3 represents a protein supernatant of the recombinant IL-18BP-Fc after fragmentation, lane 4 represents a protein precipitation of the recombinant IL-18BP-Fc after fragmentation, lane 5 represent an empty vector pET-20b(+), lane 6 represent a recombinant IL-18BP-Fc whole cell after induction at 20° C., lane 7 represents a protein supernatant of the recombinant IL-18BP-Fc after fragmentation, and lane 8 represents a protein precipitation of the recombinant IL-18BP-Fc after fragmentation.

FIGS. 2A-2C illustrate optimization of induction conditions for a target protein Sumo-IL-18BP-Fc. Specifically, in FIG. 2A: lane M represents a protein marker, lane 1 represents an empty vector pET-20b(+), and lanes 2-9 represents whole cells induced by isopropyl-β-D-thiogalactoside (IPTG) at 0 millimole per liter (mmol/L), 0.1 mmol/L, 0.3 mmol/L, 0.5 mmol/L, 0.7 mmol/L, 1 mmol/L, 1.5 mmol/L, and 2 mmol/L, respectively. In FIG. 2B: lane M represents a protein marker, lane 1 represents an empty vector pET-20b(+), and lanes 2-5 represent whole cells induced at 20° C., 30° C., and 37° C., respectively. In FIG. 2C: lane M represents a protein marker, lane 1 represents an empty vector pET-20b(+), and lanes 2-6 represent whole cells induced for 3 hours, 4 hours, 5 hours, 6 hours, and 7 hours, respectively.

FIG. 3 illustrates optimization of a fermentation process for the target protein Sumo-IL-18BP-Fc; where lane M represents a protein marker, lane 1 represents an empty vector pET-20b(+), lanes 2-12 represent whole cells cultured for 4 hours, 8 hours, 12 hours, 24 hours, 25 hours, 26 hours, 27 hours, 28 hours, 29 hours, 30 hours, and 31 hours respectively after IPTG induction, lane 13 represents a supernatant of the target protein, and lane 14 represents a precipitation of the target protein.

FIGS. 4A-4B illustrate purification results of the target protein Sumo-IL-18BP-Fc. Specifically, in FIG. 4A: lane M represents a protein marker, lane 1 represents the target protein before purification, lane 2 represents the target protein washed by 40 micromoles per liter (mM) imidazole, lane 3 represents the target protein eluted by 250 mM imidazole; in FIG. 4B: lane M represents a protein marker, lane 1 represents the target protein before purification, lane 2 represents the target protein washed by 40 mM imidazole, lane 3 represents the target protein eluted by 250 mM imidazole.

FIG. 5 illustrates results of Sumo enzyme digestion and purification of Sumo; where lane M represents a protein marker, lane 1 represents a fusion protein Sumo-IL-18BP-Fc, lane 2 represents a fusion protein Sumo-IL-18BP-Fc after enzyme digestion, lane 3 represents a protein IL-18BP-Fc purified by a nickel-nitrilotriacetic acid (Ni-NTA) column after enzyme digestion.

FIG. 6 illustrates results detected by Western Blotting, where lanes 1-3 represent the protein IL-18BP-Fc.

FIG. 7 illustrates in vitro activity detection results of the protein IL-18BP-Fc.

FIGS. 8A-8H illustrate in vivo activity detection results of the protein IL-18BP-Fc. FIGS. 8A-8B show weight changes and disease activity index monitored daily after dextran sulfate sodium salt (DSS) administration. FIG. 8C shows measured colon lengths of each group of mice sacrificed on the 15 th day. FIG. 8D shows histology scores of colonic tissues collected from mice sacrificed on the 15 th day. FIGS. 8E-8H show protein expression of designated genes of proteins extracted from the colonic tissues of mice sacrificed on the 15 th day determined by Western blotting.

FIG. 9A illustrates myeloperoxidase (MPO) activity in the colonic tissues of the mice sacrificed on the 15 th day (n=8, *p<0.05, **p<0.01).

FIG. 9B illustrates levels of aspartate aminotransferase (AST) and alanine aminotransferase (ALT) in mouse serum collected on the 15th day detected by enzyme-linked immunosorbent assay (ELISA) (n=8, *p<0.05, **p<0.01).

### DETAILED DESCRIPTION OF EMBODIMENTS

Various exemplary embodiments of the disclosure are now described in detail, which should not be considered as a limitation of the disclosure, but should be understood as a more detailed description of certain aspects, characteristics, and implementation solutions of the disclosure.

It is to be understood that the terms described in the disclosure are only intended to describe the illustrated embodiments and are not intended to limit the disclosure. Further, with respect to various value ranges in the disclosure, it is to be understood that each intermediate value between upper and lower limits of the value ranges is also specifically disclosed. Each smaller range between any stated value or an intermediate value within a stated range and any other stated value or an intermediate value within the range is also included in the disclosure. The upper and lower limits of these smaller ranges may be independently included or excluded from the scope of the disclosure.

Unless otherwise indicated, all technical and scientific terms used herein have the same meaning as commonly understood by those skilled in the related art described herein. Although the disclosure only describes illustrated methods and materials, any methods and materials similar or equivalent to those described herein may also be used in the implementation or testing of the disclosure. All literature referred to in the summary are incorporated by reference for the purpose of disclosing and describing the methods and/or materials associated with the literature. In the event of conflict with any incorporated literature, the contents of the disclosure shall prevail.

Without departing from the scope or spirit of the disclosure, it is apparent to those skilled in the art that various modifications and variations can be made to the specific embodiments of the specification of the disclosure. The other embodiments obtained from the specification of the disclosure are apparent to those skilled in the art. The specification and embodiments of the disclosure are illustrative only.

The terms “comprise”, “include”, “have”, “contain”, etc., as used herein, are open-ended terms, namely that these terms are meant to include but are not limited to.

### Embodiment 1

1. Construction of Expression Vector

According to the sequence of IL-18BP isoform a found in national center for biotechnology information (NCBI), the 3′ end of the sequence of IL-18BP isoform a is connected to the coding gene of the human immunoglobulin class G-crystallizable fragment (IgG-Fc) through the (G4S)3 linker, optimized to Escherichia coli (E. coli) preferred codons by synthesis to obtain a fusion gene of IL-18BP-Fc, then the 5′ end of the fusion gene of IL-18BP-Fc is connected to the sequence of a molecular chaperone gene Sumo to construct a fusion gene of Sumo-IL-18BP-Fc, and the fusion gene of Sumo-IL-18BP-Fc is optimized and modified. A 6×His (hexahistidine) tag is designed at the N-terminal of the recombinant protein to facilitate the subsequent purification of the recombinant protein. The recombinant plasmids pET-20b-Sumo-IL-18BP-Fc and pET-20b-IL-18BP-Fc are synthesized by Zoonbio Biotechnology Co., Ltd.

2. Transformation and Amplification of Recombinant Plasmids
1) E. coli DH5a competent cells are taken out of the refrigerator at −80° C. and dissolved on ice. Then, 5 microliters (μL) recombinant plasmids are taken and added to 100 μL DH5a competent cells and then placed in an ice bath for 30 minutes, then in a water bath at 42° C. for 90 seconds, and then in an ice bath for 1-2 minutes.
2) 1 milliliter (mL) Luria-Bertani (LB) liquid culture medium is added to the competent cells in an ultra-clean bench and shaken in a shaker at 37° C. and 200 revolutions per minute (rpm) for 1 hour.
3) The shaken mixture is centrifuged at 4000 rpm for 4 minutes, and the supernatant is discarded, leaving 200 μL bacteria solution.
4) The bacteria solution is mixed, uniformly applied on an LB solid culture medium (containing 100 micrograms per milliliter abbreviated as μg/mL Ampicillin abbreviated as Amp), and incubated at 37° C. for 12-16 hours.

3. Plasmid Extraction

The plasmid extraction is performed on the recombinant bacteria according to the instructions of a plasmid extraction kit (TIANGEN® Universal DNA Purification Kit (Spin Column)).
1) Column equilibration: 500 μL Buffer BL is added to the spin column placed in a collection tube, centrifuged at 12000 rpm for 1 minute, and the waste liquid is poured out from the collection tube.
2) 2-3 mL of bacteria solution is taken and centrifuged at 12000 rpm for 1 minute, and the supernatant is discarded.
3) 250 μL Buffer P1 is added to the precipitate obtained the step 2) of the plasmid extraction to allow the pipette to resuspend the bacteria.
4) 250 μL Buffer P2 is added to a centrifuge tube, and mixed gently upside down to fully lyse the bacteria.
5) 350 μL Buffer P3 is added to a centrifuge tube, and mixed gently upside down 4-6 times, white fluffy precipitate appears in this situation, and centrifuged at 12000 rpm for 10 minutes.
6) The supernatant obtained the step 5) of the plasmid extraction is transferred to a spin column placed in a collection tube, centrifuged at 12000 rpm for 1 minute, and the waste liquid in the collection tube is discarded.
7) 600 μL Buffer PW is added to the spin column and centrifuged at 12000 rpm for 1 minute, the waste liquid is discarded, and this step is repeated.
8) The spin column is placed into a collection tube, centrifuged at 12000 rpm for 2 minutes, and then placed at room temperature for 2 minutes.
9) The spin column is placed into a clean centrifuge tube and added 50 μL ultra-pure and sterile water (ddH₂O) in the middle of the spin column at room temperature for 2 minutes, centrifuged at 12000 rpm for 2 minutes, and the plasmid is collected in the centrifuge tube.

4. Transformation of Recombinant Plasmids into E. coli

The specific steps are the same as the method used for the transformation and amplification of the recombinant plasmids mentioned above.

5. Exploration of Expression Conditions of Recombinant Protein
1) A single transformed colony is picked and incubated overnight in 5 mL of LB liquid culture medium containing 100 μg/mL Amp in a shaking table at 160 rpm at 37° C.
2) 1 mL shaken mixture is inoculated into 100 mL of LB liquid culture medium containing Amp, incubated with shaking at 37° C. and 160 rpm.
3) When the optical density at 600 nanometers (OD₆₀₀) value of the bacteria solution is in a range of 0.6 to 0.8, isopropyl-β-D-thiogalactoside (IPTG) with a final concentration of 0.5 millimoles per liter (mmol/L) is added to induce expression, and cultured at 37° C. with shaking at 160 rpm for 4 hours.
4) The induced bacteria solution is centrifuged at 8000 rpm for 10 minutes, the supernatant is discarded, and the precipitate is resuspended with 30 mL of 20 micromoles per liter (mM) Tris-Hydrochloride (Tris-HCl) (pH 8.0) and crushed by a high-pressure homogenizer.
5) The bacteria solution before and after induction, crushed supernatant and precipitated proteins are taken and subjected to 12% sodium dodecyl sulfate-polyacrylamide gel electrophoresis (SDS-PAGE) to identify the molecular weight and soluble expression of the recombinant protein.

6. Exploration of Fermentation Process Conditions of Recombinant Protein
1) The strains with high soluble expression of the recombinant protein detected by SDS-PAGE are taken, and coated on a LB solid culture medium containing 100 μg/mL Amp with an inoculation ring, and cultured overnight at 37° C.
2) A single colony is picked and incubated overnight in 5 mL of LB liquid culture medium containing 100 μg/mL Amp in a shaking table at 160 rpm at 37° C.
3) 1 mL shaken mixture is inoculated into 100 mL of LB liquid culture medium containing Amp, incubated with shaking at 37° C. and 160 rpm.
4) Preparation of basic culture medium of a fermentation tank is as follows. 4 liters (L) basic culture medium are prepared according to the formula of 10 grams per liter (g/L) tryptone, 5 g/L yeast powder, 5 g/L NaCl, and 10 g/L anhydrous glucose, with 1 L supplement (the supplement is the same as the basic culture medium, with an adding rate of 50 milliliters per hour abbreviated as mL/h). The basic culture medium is sterilized at 121° C. for 20 minutes.
5) After the fermentation tank is pressurized, the fermentation tank is cooled to 37° C. When the OD₆₀₀ value of the seed culture medium is 1.5, 40 mL of the seed culture medium is taken and inoculated into a 10 L fermentation tank containing Amp basic culture medium, a defoamer (the concentration of defoamer in the medium of the fermentation tank is 10%) and ammonia (mass fraction was 25%) are introduced, the pH is adjusted to 7.5, and the medium is cultured at 37° C. and 200 rpm for 2 hours.
6) The temperature of the fermentation tank is reduced to 30° C. to continue cultivation. When the OD₆₀₀ value is in a range of 4.2 to 4.5, IPTG with a final concentration of 0.5 mmol/L is added to induce expression. In this situation, the temperature of the fermentation tank is reduced to 20° C., supplements are added, and the culture is continued at 400 rpm.
7) At 4, 8, 12, 24 to 31 hours after induction, the bacteria solution is taken to measure the OD₆₀₀ value and the dissolved oxygen value in the tank is recorded.
8) The bacteria solutions before and at respective times after induction are taken and subjected to 12% SDS-PAGE to detect the expression of the recombinant protein.
9) The fermented bacteria solution is taken and crushed using the same crushing method as described above.
10) The crushed supernatant and precipitated protein are taken for 12% SDS-PAGE to detect the soluble expression of the recombinant protein.

7. Purification of Recombinant Protein
1) The crushed bacteria solution is centrifuged at 4° C. for 30 minutes at 12000 rpm, the supernatant is collected, filtered with a 0.45 micrometers (μm) filter membrane to remove bacteria, and the samples are packaged and stored at −80° C.
2) An ultraviolet detector, recorder, and a peristaltic pump are turned on.
3) Column equilibration: a pre-loaded nickel-nitrilotriacetic acid (Ni-NTA) affinity chromatography column is equilibrated with 50 mM Tris-HCl buffer with pH 8.0 until the absorbance on the detector is zero.
4) Sample loading: the packaged protein samples Sumo-IL-18BP-Fc are loaded into the Ni-NTA affinity chromatography column at a flow rate of 2 milliliters per minute (mL/min). The samples are combined to the chromatography column for 30 minutes after the sample loading is completed.

5) Washing: the impurities are removed using 50 mM Trish-CI buffer containing 40 mM imidazole at a flow rate of 3 mL/min, and the impurity removal peak liquid is collected.
6) Elution: 50 mM Tris-HCl buffer containing 250 mM imidazole is used for elution, with a flow rate of 3 mL/min, and the elution peak liquid is collected.
7) Column storage: the column is eluted with 50 mM Tris-HCl buffer at pH 8.0 with a flow rate of 4 mL/min, after which the column is washed with 20% ethanol and stored in a chromatography cabinet at 4° C.
8) The molecular weight and purity of the recombinant protein are detected by 12% SDS-PAGE from the sample loading, washing, and elution liquids.

8. Enzyme Digestion and Purification of Recombinant Protein
1) The recombinant protein purified by Ni-NTA affinity chromatography column is desalted by dialysis. 50 mM Tris-HCl with the same pH as the sample to be dialyzed is used as the dialysate, and the solution of the target protein is placed in the dialysate to remove NaCl and imidazole from the purified protein.
2) Sumo enzyme and Sumo-IL-18BP-Fc protein are added to the target protein in a 2:1 ratio and digested overnight at 4° C.
3) The enzyme digestion of the recombinant protein is detected by using 12% SDS-PAGE.
4) The purification steps are the same as the purification steps of the Sumo-IL-18BP-Fc protein mentioned above, except that the collected liquid is eluate.
5) The purity of the protein IL-18BP-Fc is detected by using 12% SDS-PAGE.

9. Determination of concentration of protein IL-18BP-Fc

The concentration of the protein IL-18BP-Fc is detected according to the instructions of the Thermo Scientific™ bicinchoninic acid (BCA) protein assay kit.

10. Western Blot Detection of Target Protein
1) The purified protein is performed with SDS-PAGE. The gel is taken off and cut according to the molecular weight of the protein, and soaked in a pre-cooled transmembrane buffer solution.
2) The polyvinylidene difluoride (PVDF) membrane is activated with methanol for 10 minutes, and placed from bottom to top in the following order: black surface-sponge-3 layers of filter paper-gel-PVDF membrane-3 layers of filter paper-sponge-transparent surface. The transmembrane current is set to 300 milliamperes (mA), and the transmembrane time is set to 90 minutes.
3) After the membrane is transferred, the membrane is blocked at room temperature for 1 hour or at 4° C. overnight.
4) After the end of blocking, a first antibody incubation solution is prepared and incubated at room temperature for 1 hour or at 4° C. overnight.
5) The membrane is washed with 1× Tris-buffered saline with 0.1% Tween® 20 Detergent (TBST) for 3 times, 10 minutes each time, and a secondary antibody incubation solution is prepared and incubated at room temperature for 1 hour.
6) The membrane is washed with 1×TBST twice, 10 minutes each time, and a developer is prepared according to the instructions of the enhanced chemiluminescent (ECL) substrate kit.
7) The PVDF membrane is immersed in the developer, incubated for 1 minute, and photographed for storage.

11. Mass Spectrometry Analysis of Target Protein

The target protein is subjected to 12% SDS-PAGE, staining and decolorization, and the gel at the position of the target protein is cut off to obtain a sample. The sample is sent to Beijing Protein Innovation Co, Ltd for mass spectrometry sequencing analysis.

12. In Vitro Activity Detection of Target Protein
1) Recovery of KG-1a cells is as follows. 8 mL of Dulbecco's modified eagle medium (DMEM) is added to a 10 mL centrifuge tube; the frozen KG-1a cells are taken and melted in a 37° C. water bath; and the melted KG-1a cells are immediately added to the prepared centrifuge tube and centrifuged at 1500 rpm for 5 minutes. The supernatant is sucked out and discarded, the precipitate is re-suspended with 1 mL of complete culture medium and added to a petri dish containing 6 mL of culture medium, shaken gently, and cultured in a 5% CO 2 incubator at 37° C.
2) The KG-1a cells are taken for observation under a microscope, and once the cells are in good growth status, the KG-1a cells are spread into a 96-well plate.
3) Plating: cells are collected into a sterile centrifuge tube by using a pipette and centrifuged at 1000 rpm for 5 minutes; the supernatant is discarded, resuspended and mixed with 1 mL of complete culture medium, 1 μL mixture is taken and diluted with complete culture medium 10 times, and the cells are counted under a microscope; 100 μL KG-1a cells per well (3×10⁵ cell/mL).
4) The recombinant protein IL-18BP-Fc (0-100 nanograms per milliliter abbreviated as ng/mL) and human IL-18 (4 ng/mL) are mixed in culture medium, 100 μL mixture is added to each well and incubated in a 5% CO 2 incubator at 37° C. for 24 hours.
5) The supernatant from each well is taken to detect the content of interferon-gamma (IFN-γ) by using enzyme-linked immunosorbent assay (ELISA) kit of human IFN-γ, so as to detect the neutralization of recombinant protein IL-18BP-Fc on IL-18.

13. In Vivo Activity Detection of Target Protein

The in vivo activity of the recombinant protein IL-18BP-Fc is detected by using a mouse inflammatory bowel disease model.
1) Female C57 BL/6 mice are fed 3% dextran sulfate sodium salt (DSS) in water for 7 consecutive days to induce inflammatory bowel disease (3 grams DSS per 100 mL of water). The recombinant protein IL-18BP-Fc is injected intraperitoneally from day 0 to day 7. The changes of mouse weight and feces are monitored daily.
2) grouping experiment is performed as follows (8 mice in each group).
a. No DSS+Tris-HCl: the mice each are injected intraperitoneally with 20 mM Tris-HCl (pH 8.0) (200 μL).
b. No DSS+5 mg/kg IL-18BP-Fc: the mice each are injected intraperitoneally with recombinant protein IL-18BP-Fc (total volume 200 μL containing 5 mg/kg recombinant protein IL-18BP-Fc).
c. DSS+200 uL Tris-HCl: the mice each are injected intraperitoneally with 20 mM Tris-HCl.
d. DSS+5 mg/kg IL-18BP-Fc: the mice each are injected intraperitoneally with the recombinant protein IL-18BP-Fc (total volume 200 μL containing 5 mg/kg recombinant protein IL-18BP-Fc).
e. DSS+0.5 mg/kg IL-18BP-Fc: the mice each are injected intraperitoneally with recombinant protein IL-18BP-Fc (total volume 200 uL containing 0.5 mg/kg recombinant IL-18BP-Fc).
3) After 7 days of continuous intraperitoneal injection of the recombinant protein IL-18BP-Fc, the mice are sacrificed on the 8th day to obtain the diseased colonic tissue and detect myeloperoxidase (MPO) activity in the colonic tissue, the MPO activity is a potential marker for judging the degree of inflammation in mice, and the mice with colitis have higher MPO activity. The total protein is extracted from the tissue, the expression levels of proinflammatory cytokines IL-18, IFN-γ, IL-1β, and TNF-α are detected through western blotting, and the levels of the proinflammatory cytokines in the mice with colitis are increased.
4) After 7 days of continuous intraperitoneal injection of the recombinant protein IL-18BP-Fc, blood is taken from mice in each group on the 8^{th} day to detect the content of aspartate aminotransferase (AST) and alanine aminotransferase (ALT) in serum, and the safety of the recombinant protein IL-18BP-Fc is detected.
5) histopathology analysis is performed as follows.
a. The mice are weighed and observed for appearance (including hair, activity, and mental state) before they are sacrificed.
b. All organs (heart, liver, spleen, lungs, and kidneys) of mice are collected. After dissecting the mice, the organs were first observed by the naked eye, and compared with the control group with differences for photographic comparison.
c. 5 groups of mouse colons are collected for direct tissue fixation, Hematoxylin and Eosin (H&E) staining analysis, and histological scoring analysis.

14. Statistical Analysis

All experimental data are analyzed using the student t-test of GraphPad Prism software, and all data using Means±SEM, P<0.05 are considered to be statistically significant.

15. Results and Analysis

(1) Soluble Expression of IL-18BP-Fc Protein Promoted by Sumo

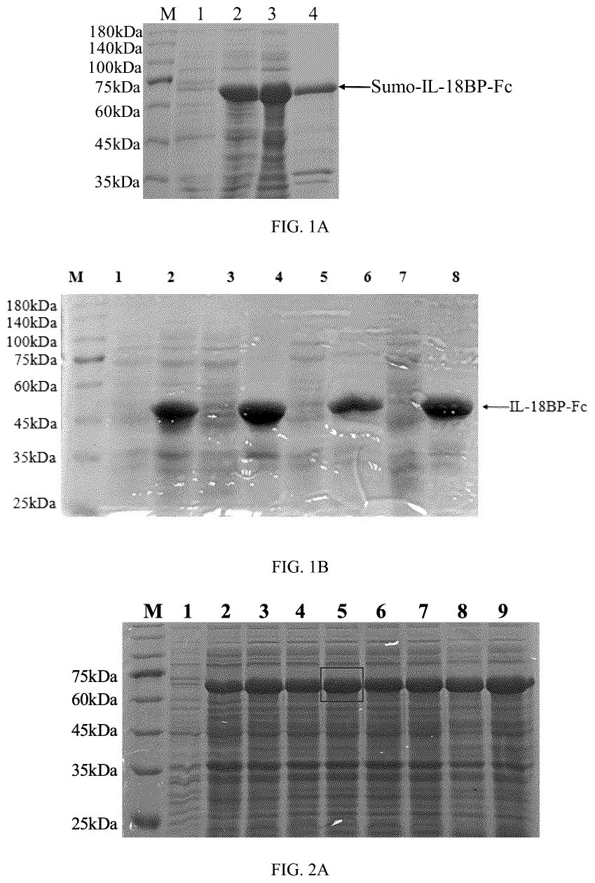

As shown in FIGS. 1A-1B, the expression of the recombinant protein Sumo-IL-18BP-Fc is induced by IPTG at 37° C. The results show that the recombinant protein Sumo-IL-18BP-Fc is mainly expressed in a soluble form (FIG. 1A). The recombinant IL-18BP-Fc bacteria are induced at 37° C. and 20° C. respectively, and the protein IL-18BP-Fc is expressed as inclusion bodies after IPTG induction (FIG. 1B).

(2) Optimization of Induction Conditions for Recombinant Protein Sumo-IL-18BP-Fc

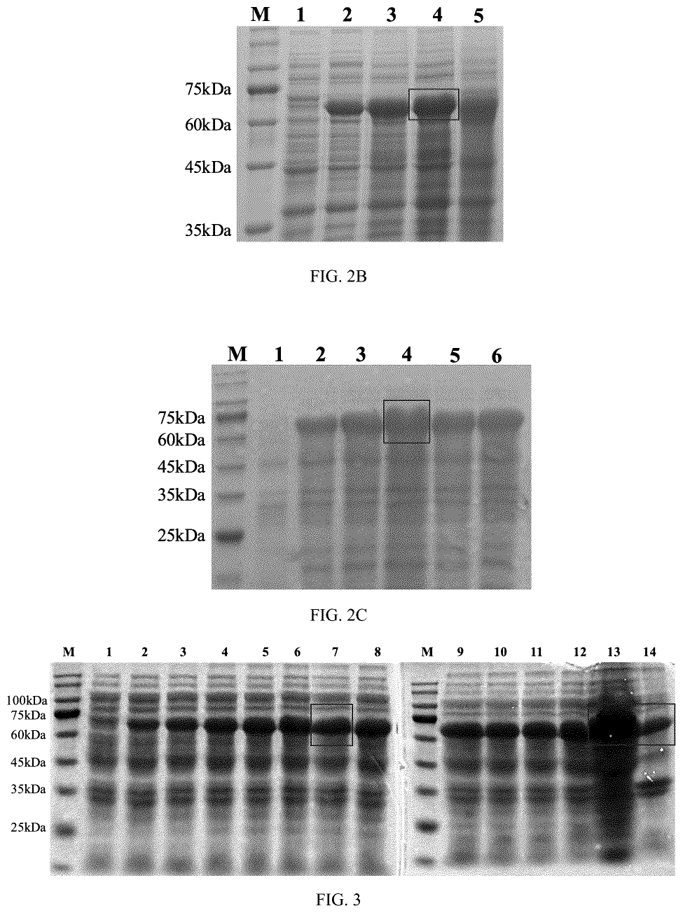

As shown in FIGS. 2A-2C, after the optimization of the IPTG induction concentration, the results show that the expression level of the target protein Sumo-IL-18BP-Fc is the highest under the induction of 0.5 mmol/L IPTG (FIG. 2A). After the optimization of the induction temperature, the results show that the expression level of the target protein Sumo-IL-18BP-Fc is the highest under the induction of 0.5 mmol/L IPTG and 30° C. (FIG. 2B). After the optimization of the induction time, the results show that the expression level of the target protein Sumo-IL-18BP-Fc is the highest expression level under the induction of 0.5 mmol/L IPTG and 30° C. for 5 hours (FIG. 2C).

(3) Optimization of Fermentation Process for Target Protein Sumo-IL-18BP-Fc

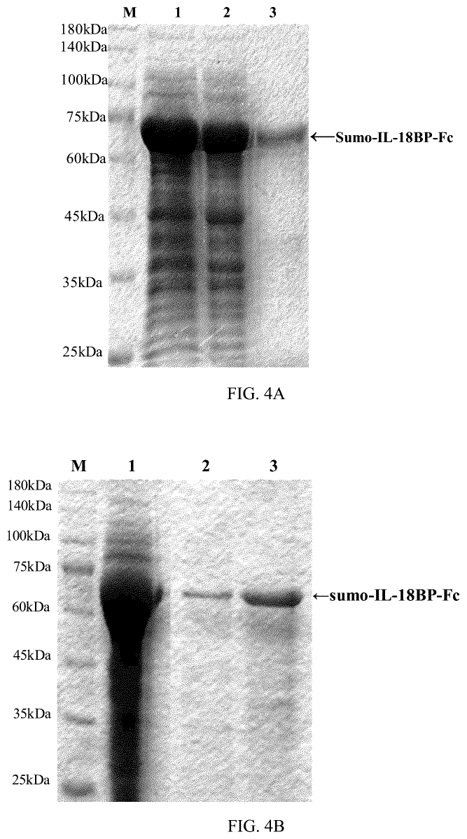

By optimizing fermentation conditions, a fermentation system with high expression of soluble target protein is obtained. Through comparison of grayscale values, it is found that the expression level of the target protein is the highest at 26 hours after induction of the target protein, and the soluble expression level of the target protein after fermentation accounted for more than 85% of the total protein (as shown in FIG. 3).

(4) Purification Results of Target Protein Sumo-IL-18BP-Fc

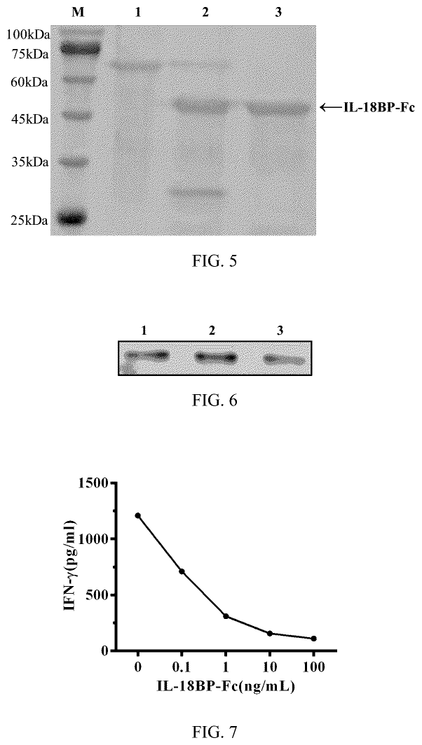

The target protein Sumo-IL-18BP-Fc is purified by the Ni-NTA affinity chromatography, and the purity is calculated to be 80% (FIG. 4A). After purification by the Ni-NTA affinity chromatography again based on the results shown in FIG. 4A, the purity is calculated to be 90% (FIG. 4B). The purified target protein Sumo-IL-18BP-Fc can be subjected to enzyme digestion.

(5) Sumo Enzyme Removal of Sumo Tag and Purification Results

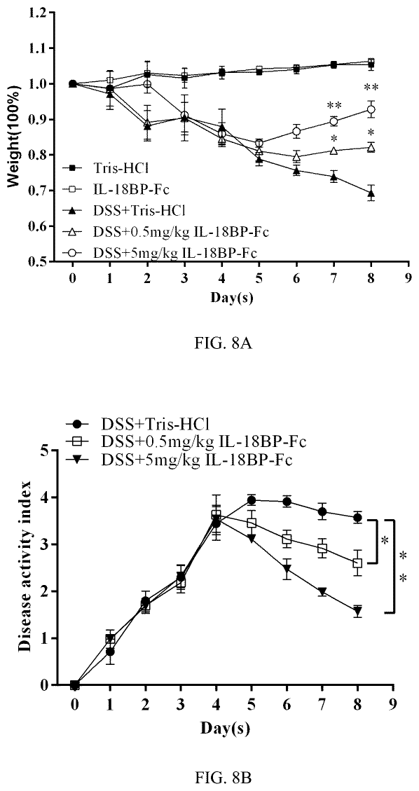

As shown in FIG. 5, based on the results in FIG. 4, the purified fusion protein Sumo-IL-18BP-Fc is digested using the Sumo enzyme to remove the Sumo tag, and then purified by the Ni-NTA affinity chromatography to obtain the protein IL-18BP-Fc. After calculation, the purity of the protein IL-18BP-Fc is 95%, and the protein concentration is 5.48 mg/mL.

(6) Results of Protein IL-18BP-Fc Detected by Western Blotting

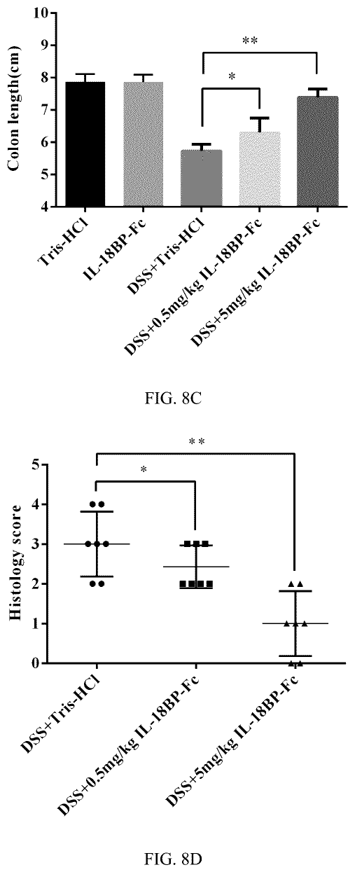

As shown in FIG. 6, the protein IL-18BP-Fc is detected by Western blotting, and the results show good expression of the protein IL-18BP-Fc after exposure to anti-Fc tag antibody detection.

(7) In Vitro Activity Detection of Protein IL-18BP-Fc

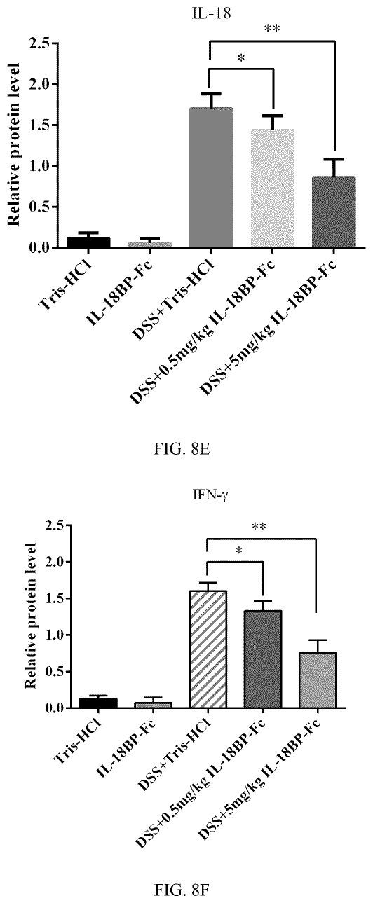

The protein IL-18BP-Fc binds to IL-18 and inhibits the secretion of IFN-γ by KG-1a cells, As shown in FIG. 7, the protein IL-18BP-Fc has good biological activity and can inhibit the secretion of IFN-γ.

(8) In Vivo Activity Detection of Protein IL-18BP-Fc

The mice are fed 3% DSS for 7 consecutive days to induce the development of inflammatory bowel disease in mice, and the activity and safety of the protein IL-18BP-Fc in vivo are detected by constructing a mouse inflammatory bowel disease model.

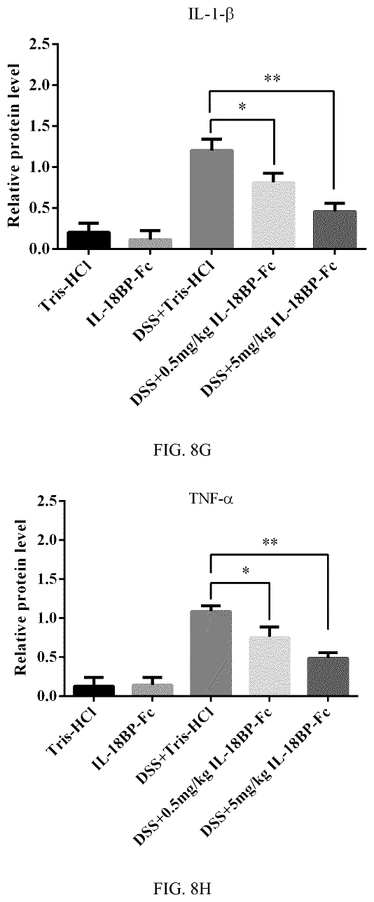

The weight changes and disease activity index are monitored daily after DSS administration, and the results are shown in FIGS. 8A-8B. Compared with the control group injected with 20 mM Tris-HCl solution, the weight loss and disease activity index of mice injected with different concentrations of IL-18BP-Fc group are significantly reduced.

After feeding 3% DSS and continuous intraperitoneal injection of different concentrations of IL-18BP-Fc for 7 days, the mice are sacrificed on the 8^{th} day, the colon lengths of the mice in each group are measured. As shown in FIG. 8C, compared with the control group, the colon lengths of mice injected with IL-18BP-Fc substantially return to normal levels.

After feeding 3% DSS and continuous intraperitoneal injection of different concentrations of IL-18BP-Fc for 7 days, the histology score results shown in FIG. 8D show that compared with the control group, the crypt loss, epithelial damage and inflammation in the colon of the mice injected with different concentrations of IL-18BP-Fc are reduced, indicating that IL-18BP-Fc can repair colitis damage in mice.

After the mice are sacrificed on the 8th day, colonic tissue is collected and subjected to Western blot analysis. As shown in FIG. 8E, FIG. 8F, FIG. 8G, and FIG. 8H, after the mice are sacrificed, proteins are extracted from the colonic tissue homogenate, the protein expressions of IL18, IFN-γ, IL1β and TNF-α are measured by Western blotting, and the results show that the protein expressions of IL18, IFN-γ, IL1β and TNF-α are decreased in the experimental group after the injection of IL-18BP-Fc.

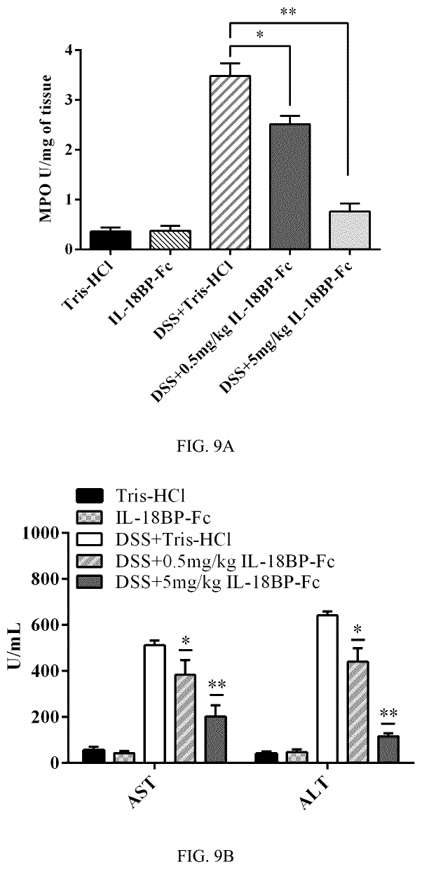

After the mice are sacrificed on the 8th day, colonic tissue samples are collected to evaluate tissue MPO activity. As shown in FIG. 9A, compared with the control group, the MPO activity of the colonic tissue in mice is significantly reduced after the injection of IL-18BP-Fc, indicating improved colitis in mice.

On the 8^{th} day, the serum of each group of mice is collected and the AST and ALT levels in the serum are detected by ELISA. As shown in FIG. 9B, compared with the control group, the AST and ALT levels in the serum of mice are significantly reduced after injection of IL-18BP-Fc, indicating that IL-18BP-Fc has good safety.

The above results demonstrate that after administering DSS to induce inflammatory colitis, intraperitoneal injection of IL-18BP-Fc can effectively neutralize IL-18 and inhibit disease development.

The above embodiments are merely a description of the illustrated method of the disclosure and are not intended to limit the scope of the disclosure. Without departing from the design spirit of the disclosure, various modifications and changes made by those skilled in the art to the technical solutions of the disclosure shall fall within the scope of protection determined in the claims of the disclosure.

What is claimed is:

1. A method for fermenting and producing a recombinant interleukin-18 binding protein (IL-18BP), comprising:
inducing fermentation to culture a host bacterium to obtain the recombinant interleukin-18 binding protein;
wherein expression conditions for the inducing with isopropyl-β-D-thiogalactoside (IPTG) are as follows: induction at 0.5 millimoles per liter (mmol/L) IPTG at 20 Celsius degree (° C.) for 4-31 hours;
wherein the recombinant IL-18BP is prepared as follows:
connecting a 5′ end of a coding gene of the recombinant IL-18BP with a gene sequence of a molecular chaperone Sumo to construct a fusion gene, inserting the fusion gene into a cell expression vector, and guiding the cell expression vector into a prokaryotic cell to express the recombinant IL-18BP;
wherein the recombinant IL-18BP comprises a sequence encoding human IL-18BP isoform a (hIL-18BPa) and a sequence encoding human immunoglobulin class G-crystallizable fragment (IgG-Fc); an amino acid sequence of the recombinant IL-18BP is shown in SEQ ID NO: 1, a nucleotide sequence of the coding gene of the recombinant IL-18BP is shown in SEQ ID NO: 3;
and an amino acid sequence of the molecular chaperone Sumo is shown in SEQ ID NO: 2.

2. The method according to claim 1, wherein the cell expression vector comprises pET-20b(+).
